(19) United States
(12) Patent Application Publication
(10) Pub. No.: US 2024/0011024 A1
(43) Pub. Date: Jan. 11, 2024

(54) CRISPR SPACER TAGS FOR LABELING AND/OR IDENTIFYING BACTERIA, AND METHODS OF USING THE SAME

(71) Applicant: North Carolina State University, Raleigh, NC (US)

(72) Inventor: Rodolphe Barrangou, Raleigh, NC (US)

(21) Appl. No.: 18/251,636

(22) PCT Filed: Nov. 4, 2021
(86) PCT No.: PCT/US2021/057978

§ 371 Date: May 3, 2023

Related U.S. Application Data
(60) Provisional application No. 63/109,547, filed on Nov. 4, 2020.

(51) Int. Cl. C12N 15/11; C12Q 1/689

(52) U.S. Cl. CPC C12N 15/11; C12Q 1/689; C12N 2310/20

(57) ABSTRACT

This invention relates to synthetic CRISPR spacer polynucleotides and compositions comprising the same such as synthetic spacer-repeat sequences and synthetic CRISPR arrays, wherein the synthetic CRISPR spacer polynucleotides, when translated according to amino acid single-letter code convention, spell a text. Further provided in this invention are methods of using the same for labeling and/or detecting bacteria of interest.

### STATEMENT OF PRIORITY

This application claims the benefit, under 35 U.S.C. § 119(e), of U.S. Provisional Application No. 63/109,547, filed on Nov. 4, 2020, the entire contents of which are incorporated by reference herein.

### STATEMENT REGARDING ELECTRONIC FILING OF A SEQUENCE LISTING

A Sequence Listing in ASCII text format, submitted under 37 C.F.R. § 1.821, entitled 5051-984WO_ST25.txt, 2,600 bytes in size, generated on Oct. 30, 2021 and filed via EFS-Web, is provided in lieu of a paper copy. This Sequence Listing is hereby incorporated herein by reference into the specification for its disclosures.

### FIELD OF THE INVENTION

This invention relates to synthetic CRISPR spacer polynucleotides, synthetic CRISPR arrays, compositions comprising the same, and methods of using the same for labeling and/or detecting bacteria of interest.

### BACKGROUND OF THE INVENTION

Clustered Regularly Interspaced Short Palindromic Repeats (CRISPR), in combination with CRISPR-associated genes (cas) constitute the CRISPR-Cas system, which confers adaptive immunity in many bacteria and most archaea. CRISPR-mediated immunization occurs through the integration of DNA from invasive genetic elements such as plasmids and phages that can be used to thwart future infections by invaders containing the same sequence.

CRISPR-Cas systems consist of CRISPR arrays of short DNA “repeats” interspaced by hypervariable “spacer” sequences and a set of flanking cas genes. The system acts by providing adaptive immunity against invasive genetic elements such as phage and plasmids through the sequence-specific targeting and interference of foreign nucleic acids (Barrangou et al. 2007. Science. 315:1709-1712; Brouns et al. 2008. Science 321:960-4; Horvath and Barrangou. 2010. Science. 327:167-70; Marraffini and Sontheimer. 2008. Science. 322:1843-1845; Bhaya et al. 2011. Annu. Rev. Genet. 45:273-297; Terns and Terns. 2011. Curr. Opin. Microbiol. 14:321-327; Westra et al. 2012. Annu. Rev. Genet. 46:311-339; Barrangou R. 2013. RNA. 4:267-278). Typically, invasive DNA sequences are acquired as novel “spacers” (Barrangou et al. 2007. Science. 315:1709-1712), each paired with a CRISPR repeat and inserted as a novel repeat-spacer unit in the CRISPR locus. The “spacers” are acquired by the Cas1 and Cas2 proteins that are universal to all CRISPR-Cas systems (Makarova et al. 2011. Nature Rev. Microbiol. 9:467-477; Yosef et al. 2012. Nucleic Acids Res. 40:5569-5576), with involvement by the Cas4 protein in some systems (Plagens et al. 2012. J. Bact. 194: 2491-2500; Zhang et al. 2012. PLoS One 7:e47232). The resulting repeat-spacer array is transcribed as a long pre-CRISPR RNA (pre-CRISPR, pre-crRNA) (Brouns et al. 2008. Science 321:960-4), which is processed into CRISPR RNAs (CRISPRs, crRNAs) that drive sequence-specific recognition of DNA or RNA. Specifically, crRNAs guide nucleases towards complementary targets for sequence-specific nucleic acid cleavage mediated by Cas endonucleases (Garneau et al. 2010. Nature. 468:67-71; Haurwitz et al. 2010. Science. 329:1355-1358; Sapranauskas et al. 2011. Nucleic Acid Res. 39:9275-9282; Jinek et al. 2012. Science. 337:816-821; Gasiunas et al. 2012. Proc. Natl. Acad. Sci. 109:E2579-E2586; Magadan et al. 2012. PLoS One. 7:e40913; Karvelis et al. 2013. RNA Biol. 10:841-851). These widespread systems occur in nearly half of bacteria (about 46%) and the large majority of archaea (about 90%). CRISPR/Cas are subdivided in classes and types based on the cas gene content, organization and variation in the biochemical processes that drive crRNA biogenesis, and Cas protein complexes that mediate target recognition and cleavage. Class 1 uses multiple Cas proteins in a cascade complex to degrade nucleic acids. Class 2 uses a single large Cas protein to degrade nucleic acids.

### SUMMARY OF THE INVENTION

A first aspect of the present invention provides a synthetic CRISPR spacer polynucleotide having a 5′ end and a 3′ end and comprising a spacer tag having a length of about 20 to about 50 nucleotides, which when translated according to amino acid single-letter code convention spells a text, wherein the synthetic CRISPR spacer polynucleotide is non-functional. In some embodiments, the spacer polynucleotide is non-transcribable, non-interfering, and/or non-targeting. In some embodiments, the text is a word, an abbreviation, and/or an acronym. In some embodiments, the spacer polynucleotide is linked at its 5′ end to a repeat sequence to form a repeat-spacer sequence.

A second aspect of the present invention provides a synthetic CRISPR array comprising a leader end (e.g., 5′ portion), a trailer end (e.g., 3′ portion), and at least one repeat-spacer sequence (e.g., a repeat-spacer sequence of the present invention), wherein the at least one repeat-spacer sequence comprises a 5′ end and a 3′ end and the 3′ end of the repeat spacer sequence is linked to a repeat sequence (e.g., to form a repeat-spacer-repeat sequence).

Another aspect of the present invention provides a vector comprising the synthetic CRISPR spacer polynucleotide and/or synthetic CRISPR array of the present invention.

Another aspect of the present invention provides a bacterium comprising the synthetic CRISPR spacer polynucleotide and/or synthetic CRISPR array of the present invention.

A further aspect of the present invention provides a method of labeling a bacterial cell, comprising: (a) introducing into the bacterial cell a synthetic CRISPR spacer polynucleotide of the present invention (e.g., a repeat-spacer sequence of the present invention) or a vector comprising the synthetic CRISPR spacer polynucleotide of the present invention into an endogenous/native CRISPR array of the bacterial cell, the endogenous CRISPR array comprising a leader end (5′ portion) and a trailer end (3′ portion), wherein the synthetic CRISPR spacer polynucleotide is introduced into/at the trailer end of the CRISPR array; and/or (b) introducing into the bacterial cell a synthetic CRISPR array of the present invention or a vector comprising the synthetic CRISPR array of the present invention; thereby labeling the bacterial cell. In some embodiments, the synthetic CRISPR spacer polynucleotide of the present invention is inserted into/at the trailer end (3′ portion) of the endogenous/native CRISPR array.

Another aspect of the present invention provides a method of detecting a bacterial cell, comprising: (a) introducing into the bacterial cell (i) a synthetic CRISPR spacer polynucleotide of the present invention (e.g., a repeat-spacer sequence of the present invention) or a vector comprising the synthetic CRISPR spacer polynucleotide of the present invention into an endogenous/native CRISPR array of the bacterial cell, the endogenous CRISPR array comprising a leader end (5′ portion) and a trailer end (3′ portion), wherein the synthetic CRISPR spacer polynucleotide is introduced into/at the trailer end of the CRISPR array; and/or (ii) introducing into the bacterial cell a synthetic CRISPR array of the present invention or a vector comprising the synthetic CRISPR array of the present invention, thereby labeling the bacterial cell; and (b) detecting the presence of the synthetic polynucleotide, thereby detecting the bacterial cell.

In some embodiments, a bacterial cell of the present invention may include a lactic acid bacterial cell, a probiotic bacterial cell, an industrial fermentation bacterial cell, a microbiome bacterial cell, a biotherapeutic bacterial cell, pathogenic bacterial cell, a commensal bacterial cell, a spoilage bacterial cell (e.g., food spoilage), and/or any combination thereof.

Further provided are the recombinant cells and/or organisms produced by the methods of the invention and nucleic acid constructs for carrying out the methods. These and other aspects of the invention are set forth in more detail in the description of the invention below.

### BRIEF DESCRIPTION OF THE DRAWINGS

FIG. 1 provides a schematic of the generic structure of a CRISPR array comprising a 5′ leader end, a 3′ trailer end, one or more CRISPR spacers and one or more CRISPR repeats.

FIGS. 2A-2B provide schematics describing (FIG. 2A) the addition of new spacers into/at the leader end of a natural (endogenous) CRISPR array, for example, when exposed to phage; and in comparison (FIG. 2B) introduction of synthetic CRISPR spacer tag (“CRISPR-tag”) into/at the trailer end of a CRISPR array, for example, an endogenous array.

FIG. 3 provides (FIG. 3, top) a table of the amino acid single-letter code convention and (FIG. 3, bottom) an example of a synthetic CRISPR spacer polynucleotide comprising a CRISPR tag (“CRISPR-tag”), introduced into/at the trailer end of the CRISPR array, and which, when translated according to amino acid single-letter code convention, spells a text (“CRISPRTAG”; SEQ ID NO:1). The nucleic acid sequence shown depicts

(SEQ ID NO: 2)
TGTCGTATTTCTCCTAGAACTGCTGGT.

FIGS. 4A-4B show a spacer sequence design comprising a CRISPR-tag spacer flanked between two repeats (repeat 1 and repeat 2). FIG. 4A shows a schematic of a plasmid comprising the spacer. FIG. 4B shows the sequencing results of the inset box of FIG. 4A.

FIGS. 5A-5B show a spacer sequence design comprising a CRISPR-tag spacer with no flanking repeats. FIG. 5A shows a schematic of a plasmid comprising the spacer. FIG. 5B shows the sequencing results of the inset box of FIG. 5A.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The terms “complementary” or “complementarity,” as used herein, refer to the natural binding of polynucleotides under permissive salt and temperature conditions by base-pairing. For example, the sequence “A-G-T” binds to the complementary sequence “T-C-A.” Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement” as used herein can mean 100% complementarity with the comparator nucleotide sequence or it can mean less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

As used herein, the phrase “substantially complementary,” or “substantial complementarity” in the context of two nucleic acid molecules, nucleotide sequences or protein sequences, refers to two or more sequences or subsequences that are at least about 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue complementary, when compared and aligned for maximum correspondence, as measured using one of the following sequence comparison algorithms or by visual inspection. In some embodiments, substantial complementarity can refer to two or more sequences or subsequences that have at least about 80%, at least about 85%, at least about 90%, at least about 95, 96, 96, 97, 98, or 99% complementarity (e.g., about 80% to about 90%, about 80% to about 95%, about 80% to about 96%, about 80% to about 97%, about 80% to about 98%, about 80% to about 99% or more, about 85% to about 90%, about 85% to about 95%, about 85% to about 96%, about 85% to about 97%, about 85% to about 98%, about 85% to about 99% or more, about 90% to about 95%, about 90% to about 96%, about 90% to about 97%, about 90% to about 98%, about 90% to about 99% or more, about 95% to about 97%, about 95% to about 98%, about 95% to about 99% or more). Two nucleotide sequences can be considered to be substantially complementary when the two sequences hybridize to each other under stringent conditions. In some representative embodiments, two nucleotide sequences considered to be substantially complementary hybridize to each other under highly stringent conditions.

As used herein, “contact,” contacting,” “contacted,” and grammatical variations thereof, refers to placing the components of a desired reaction together under conditions suitable for carrying out the desired reaction (e.g., integration, transformation, site-specific cleavage (nicking, cleaving), amplifying, site specific targeting of a polypeptide of interest and the like). The methods and conditions for carrying out such reactions are well known in the art (See, e.g., Gasiunas et al. (2012) Proc. Natl. Acad. Sci. 109:E2579-E2586; M. R. Green and J. Sambrook (2012) Molecular Cloning: A Laboratory Manual. 4th Ed., Cold Spring Harbor Laboratory Press, Cold Spring Harbor, NY).

As used herein, the term “commensal bacteria” refers to a bacterium that is naturally present in a microbiome, such as in the gut microbiome of a host (e.g., human gut microbiome), without causing harm to the host. In some cases, a commensal bacterium may confer a benefit to the host organism.

A “fragment” or “portion” of a nucleic acid will be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising a nucleotide sequence of contiguous nucleotides that are identical or almost identical (e.g., 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the invention may be, where appropriate, included in a larger polynucleotide of which it is a constituent.

A “heterologous” or a “recombinant” nucleic acid is an exogenous nucleic acid not naturally associated with a host cell into which it is introduced, including non-naturally occurring multiple copies of a naturally occurring nucleic acid. In some embodiments, “heterologous” may include a nucleic acid that is endogenous to a host cell but is in a non-natural position relative to the wild type as a result of human intervention.

As used herein, hybridization, hybridize, hybridizing, and grammatical variations thereof, refer to the binding of two complementary nucleotide sequences or substantially complementary sequences in which some mismatched base pairs are present. The conditions for hybridization are well known in the art and vary based on the length of the nucleotide sequences and the degree of complementarity between the nucleotide sequences. In some embodiments, the conditions of hybridization can be high stringency, or they can be medium stringency or low stringency depending on the amount of complementarity and the length of the sequences to be hybridized. The conditions that constitute low, medium and high stringency for purposes of hybridization between nucleotide sequences are well known in the art (See, e.g., Gasiunas et al. (2012) Proc. Natl. Acad. Sci. 109:E2579-E2586; M. R. Green and J. Sambrook (2012) Molecular Cloning: A Laboratory Manual. 4th Ed., Cold Spring Harbor Laboratory Press, Cold Spring Harbor, NY).

A “native” or “wild type” nucleic acid, nucleotide sequence, polypeptide or amino acid sequence refers to a naturally occurring or endogenous nucleic acid, nucleotide sequence, polypeptide, or amino acid sequence. Thus, for example, a “wild type CRISPR array” is a CRISPR array that is naturally occurring in or endogenous to the organism. An “endogenous” nucleic acid is a nucleic acid naturally associated with a host cell into which it is introduced.

As used herein, the terms “nucleic acid,” “nucleic acid molecule,” “nucleic acid construct,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The nucleic acid constructs of the present disclosure can be DNA or RNA. In some embodiments, the nucleic acid constructs of the present disclosure are DNA. Thus, although the nucleic acid constructs of this invention may be described and used in the form of DNA, depending on the intended use, they may also be described and used in the form of RNA.

A “synthetic” nucleic acid or nucleotide sequence, as used herein, refers to a nucleic acid or nucleotide sequence that is not found in nature but is constructed by human intervention, and as a consequence, it is not a product of nature.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide,” and “polynucleotide” are also used interchangeably herein to refer to a heteropolymer of nucleotides. Except as otherwise indicated, nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end. Thus, for example, an element in the 3′ region of a polynucleotide can be located anywhere from the first nucleotide located at the 3′ end of the polynucleotide to the nucleotide located halfway through the polynucleotide. An element that is described as being “at the 5′end” or “at the 3′end” of a polynucleotide (5′ to 3′) refers to an element located immediately adjacent to (upstream of) the first nucleotide at the 5′ end of the polynucleotide, or immediately adjacent to (downstream of) the last nucleotide located at the 3′ end of the polynucleotide, respectively.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

A “CRISPR” as used herein comprises one or more repeat sequences and one or more spacer sequence(s), wherein each of the one or more spacer sequences is linked at least at its 5′-end to a repeat sequence or portion thereof. A “CRISPR” as used herein can include a CRISPR array, an unprocessed CRISPR, or a mature/processed CRISPR or a CRISPR that comprises one repeat, or a portion thereof, and a spacer (e.g., repeat-spacer). A “CRISPR array” as used herein refers to a nucleic acid molecule that comprises at least two CRISPR repeat sequences, or a portion(s) thereof, and at least one spacer sequence, wherein one of the two repeat sequences, or a portion thereof, is linked to the 5′ end of the spacer sequence and the other of the two repeat sequences, or portion thereof, is linked to the 3′ end of the spacer sequence. In some embodiments, in a recombinant CRISPR (e.g., synthetic CRISPR array) of the invention, the combination of repeat nucleotide sequences and spacer sequences is synthetic and not found in nature. A CRISPR may be introduced into a cell or cell free system as RNA, or as DNA in an expression cassette or vector (e.g., plasmid, bacteriophage). A synthetic CRISPR array can also include an endogenous CRISPR array into which a synthetic CRISPR spacer polynucleotide of the invention is introduced.

A CRISPR useful with this invention is as defined herein and may be an unprocessed or a processed (e.g., mature) CRISPR or a CRISPR that is non-natural (e.g., repeat-spacer). A processed CRISPR comprises a spacer linked at its 5′ end and its 3′ end to a repeat sequence, wherein the repeat sequence is a portion of a full length repeat sequence. An unprocessed CRISPR comprises at least one spacer linked at both its 5′ end and its 3′ end to a full length repeat sequence. A “non-natural CRISPR” or synthetic CRISPR, as used herein, refers also to a CRISPR comprising a spacer (e.g., a non-native spacer) that has not previously been acquired by the target bacterial cell (e.g., the bacterial cell for which tagging is desired).

As used herein, the term “spacer” or “spacer sequence” refers to a nucleotide sequence that, in a native CRISPR system, is complementary to a targeted portion (i.e., “protospacer”) of a nucleic acid or a genome. The term “genome,” as used herein, refers to both chromosomal and non-chromosomal elements (i.e., extrachromosomal (e.g., mitochondrial, plasmid, a chloroplast, and/or extrachromosomal circular DNA (eccDNA))) of a target organism. A native spacer sequence functions as a guide for the CRISPR machinery to the targeted portion of the genome, wherein the targeted portion of the genome may be, for example, modified (e.g., a deletion, an insertion, a single base pair addition, a single base pair substitution, a single base pair removal, a stop codon insertion, and/or a conversion of one base pair to another base pair (base editing)). As another example, the native spacer sequence may be used to guide the CRISPR machinery to the targeted portion of the genome, wherein the targeted portion of the genome may be cut and degraded, thereby killing the cell(s) comprising the target sequence. To perform these functions, a native CRISPR spacer sequence (e.g., a functioning CRISPR spacer) needs to be transcribable/transcribing, targeting (complementary to a targeted portion), and/or interfering (initiating the degradation of a targeted portion).

As used herein, the term “synthetic spacer” or “synthetic CRISPR spacer polynucleotide” refers to a non-natural spacer sequence comprising the physical characteristics (e.g., approximate length, e.g., position within a CRISPR array when introduced into an array (e.g., between two repeat sequences)) of a natural CRISPR sequence but which sequence is not found in nature. In some embodiments, a synthetic spacer of the present invention may be non-functional, i.e., may not be able to function as a native or a natural CRISPR spacer as defined herein. A synthetic CRISPR spacer polynucleotide of the present invention may comprise a “spacer tag.”

As used herein, the term “spacer tag” refers to a nucleotide sequence comprised within a spacer sequence (e.g., a synthetic CRISPR spacer polynucleotide), which when translated according to amino acid single-letter code convention spells a text, such as a word, an abbreviation, and/or an acronym. A spacer tag of the present invention provides the ability to tag, track, and detect discrete bacteria of interest within which a synthetic CRISPR spacer polynucleotide comprising a spacer tag has been introduced.

As used herein, the terms “target genome” or “targeted genome” refer to a genome of an organism of interest.

A “target sequence” or “protospacer” refers to a targeted portion of a genome or of a cell free nucleic acid that is complementary to a spacer sequence of a native CRISPR. A target sequence or protospacer may be located immediately adjacent to a PAM (protospacer adjacent motif) (e.g., 5′-PAM-Protospacer-3′).

CRISPR Cas systems useful with this invention for tagging/labeling, tracking, and/or detecting a bacterium or a bacterial population include, but are not limited to, a Type I CRISPR-Cas system, a Type II CRISPR-Cas system, a Type III CRISPR-Cas system, a Type IV CRISPR-Cas system, a Type V CRISPR-Cas system or a Type VI CRISPR-Cas system.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the phrase “substantially identical,” or “substantial identity” in the context of two nucleic acid molecules, nucleotide sequences or protein sequences, refers to two or more sequences or subsequences that have at least about 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity can refer to two or more sequences or subsequences that have at least about 80%, at least about 85%, at least about 90%, at least about 95, 96, 96, 97, 98, or 99% sequence identity.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

In some embodiments, the polynucleotides and polypeptides of the invention are “isolated.” An “isolated” polynucleotide sequence or an “isolated” polypeptide is a polynucleotide or polypeptide that, by human intervention, exists apart from its native environment and is therefore not a product of nature. An isolated polynucleotide or polypeptide may exist in a purified form that is at least partially separated from at least some of the other components of the naturally occurring organism or virus, for example, the cell or viral structural components or other polypeptides or nucleic acids commonly found associated with the polynucleotide. In representative embodiments, the isolated polynucleotide and/or the isolated polypeptide may be at least about 1%, 5%, 10%, 20%, 30%, 40%, 50%, 60%, 70%, 80%, 90%, 95%, or more pure.

In other embodiments, an isolated polynucleotide or polypeptide may exist in a non-natural environment such as, for example, a recombinant host cell. Thus, for example, with respect to nucleotide sequences, the term “isolated” means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur (e.g., a different host cell, different regulatory sequences, and/or different position in the genome than as found in nature). Accordingly, the polynucleotides and their encoded polypeptides are “isolated” in that, through human intervention, they exist apart from their native environment and therefore are not products of nature, however, in some embodiments, they can be introduced into and exist in a recombinant host cell.

By “operably linked” or “operably associated” as used herein, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

### Compositions

The present invention relates to the design of a non-natural nucleic acid tag (spacer tag) comprising and/or comprised within a CRISPR spacer sequence, and the use of the synthetic spacer tag to label bacteria of interest. The present invention provides synthetic CRISPR spacer polynucleotides, synthetic CRISPR arrays, vectors, and bacteria comprising the same, and methods of using the same.

The present invention provides the ability to tag, track, and detect discrete bacteria of interest, such as for example, tagging and tracking proprietary clones and/or strains for use in commercial products, industrial settings (e.g., fermentation), and clinical samples. While not wishing to be bound to theory, the compositions and methods of the present invention provide the ability to tag, track, and detect bacteria of interest notably in a biologically safe mechanism. For example, the spacer tag and/or the synthetic CRISPR spacer polynucleotide is non-functional, e.g., non-transcribing, non-interfering, and/or non-targeting. Furthermore, when introduced into a CRISPR array (either synthetic and/or endogenous/native), the synthetic CRISPR spacer polynucleotide may be introduced into the trailer end (3′ portion, e.g., minimally to non-transcribed portion) of the CRISPR array. Furthermore, while not wishing to be bound to theory, the location of the non-functional spacer tag within a synthetic and/or endogenous/native CRISPR array within a bacterium's native CRISPR system, a location (spacer sequence) which natively functions as a sequence-specific target of foreign nucleic acids, minimizes the ability of the spacer tag being recognized by the bacterium's defense systems (e.g., protected through endogenous mechanisms that reduce self-targeting by CRISPR systems) and/or otherwise behaving erroneously within the cellular machinery of the bacterial cell.

Thus, one aspect of the invention relates to a synthetic CRISPR spacer polynucleotide having a 5′ end and a 3′ end and comprising a spacer tag which when translated according to amino acid single-letter code convention spells a text, wherein the synthetic CRISPR spacer polynucleotide is non-functional.

As used herein, the term “non-functional” refers to the inability of a synthetic and/or modified (recombinant) component to function as a corresponding native or any natural component, e.g., a synthetic CRISPR spacer polynucleotide not able to function as a native or any natural CRISPR spacer. There may be multiple mechanisms by which a synthetic CRISPR spacer polynucleotide of the present invention may be non-functional, i.e., non-performing of the biological mechanisms of a native/endogenous spacer sequence. For example, in some embodiments, a synthetic CRISPR spacer polynucleotide may be non-functional because it is non-transcribing, e.g., does not comprise a promoter and/or is positioned too distal from a promoter to be substantially transcribed (for example, when introduced into an endogenous CRISPR array). In some embodiments a synthetic CRISPR spacer polynucleotide may non-functional because it is non-targeting, e.g., does not comprise a sequence that targets a sequence comprised in the genome of a bacterium or archaeon of interest. In some embodiments a synthetic CRISPR spacer polynucleotide may be non-functional because it is non-interfering, e.g., does not bind to a target sequence in such a way as to initiate degradation of the target sequence in the genome of a bacterium or archaeon of interest.

In some embodiments, the synthetic CRISPR spacer polynucleotide may comprise a spacer tag having a length of about 20 to about 40 nucleotides (e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides or any value or range therein). For example, the spacer tag may have a length of about 20 nucleotides to about 36 nucleotides, about 30 nucleotides to about 40 nucleotides, about 25 nucleotides to about 38 nucleotides, about 20 to about 50 nucleotides, or about 30 nucleotides to about 36 nucleotides (e.g., about 20, 21, 22, 23, 24, 25, 26, 27, 28, or 29 nucleotides to about 30, 31, 32, 33, 34, 35, 36, 37, 38, 39, or 40 nucleotides or any value or range therein) Although amino acid residues are transcribed by nucleotides in multiples of 3, the number of nucleotides in a spacer tag and/or in a synthetic CRISPR spacer polynucleotide may, but does not need to be, a multiple of 3. In other words, a spacer tag and/or synthetic CRISPR spacer polynucleotide of the present invention may have a length of nucleotides in multiples of 3, plus at least one or at least two additional nucleotides, which, due to there being less than 3 nucleotides, would not transcribe an amino acid and thus not alter the resultant encoded tag text.

In some embodiments, the synthetic CRISPR spacer polynucleotide (e.g., inclusive of the spacer tag) comprises a length of about 20 nucleotides to about 100 nucleotides, e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides or any value or range therein. For example, in some embodiments, the synthetic CRISPR spacer polynucleotide may comprise a length of about 20 nucleotides to about 50 nucleotides, about 20 nucleotides to about 60 nucleotides, about 20 nucleotides to about 80 nucleotides, about 25 nucleotides to about 100 nucleotides, about 30 nucleotides to about 36 nucleotides, about 20 nucleotides to about 40 nucleotides, or about 26 nucleotides to about 60 nucleotides (e.g., about 20, 21, 22, 23, 24, 25, 26, 27, 28, 29 or 30 nucleotides to about 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides; e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 nucleotides to about 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides). In some embodiments, the synthetic CRISPR spacer polynucleotide comprises a length greater than the length of the spacer tag. In some embodiments, the synthetic CRISPR spacer polynucleotide comprises, consists essentially of, or consists of the spacer tag.

In some embodiments, the synthetic CRISPR spacer polynucleotide comprises a length greater than the length of the spacer tag. For example, in some embodiments, the synthetic CRISPR spacer polynucleotide may comprise a length that is greater than the length of the spacer tag by about 1 to about 60 nucleotides, e.g., about 1 to about 60 nucleotides additional to the nucleotides of the spacer tag (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 additional nucleotides).

In some embodiments, wherein the synthetic CRISPR spacer polynucleotide comprises a length greater than the length of the spacer tag, the spacer tag is located at either the 5′ end, at the 3′ end, or any position within the synthetic CRISPR spacer polynucleotide. In some embodiments, e.g., optionally wherein the spacer tag happens to share complementarity to a region in a target organism, the synthetic CRISPR spacer polynucleotide comprises a length greater than the length of the spacer tag and the spacer tag is located at the 5′ end or the 3′ end of the CRISPR spacer polynucleotide, the 5′ and or the 3′ end being the farthest from any protospacer adjacent motif (PAM) sequence in the target organism.

In some embodiments, a synthetic CRISPR spacer polynucleotide of the present invention comprises a spacer tag which when translated according to amino acid single-letter code convention spells a text. In some embodiments, the text is a word, an abbreviation, and/or an acronym. In some embodiments, the text may comprise, consist of, or consist essentially of a word, abbreviation and/or acronym, which is not naturally found in the organism to be tagged, e.g., wherein the spacer tag is a non-natural nucleic acid in the organism to be tagged. In some embodiments, the text that is spelled when the spacer tag is translated according to amino acid single-letter code convention may comprise a word, abbreviation, and/or acronym including, but not limited to, TAG, TECH (SEQ ID NO:3), DETECT (SEQ ID NO:4), CRTECH (SEQ ID NO:5), CRTAG (SEQ ID NO:6), CRDETECT (SEQ ID NO:7), CRISPR (SEQ ID NO:8), or any combination thereof. In some embodiments, the text that is spelled when the spacer tag is translated according to amino acid single-letter code convention may comprise a word, abbreviation, and/or acronym including, but not limited to, TAG, TECH (SEQ ID NO:3), DETECT (SEQ ID NO:4), CRTECH (SEQ ID NO:5), CRTAG (SEQ ID NO:6), CRDETECT (SEQ ID NO:7), CRISPR (SEQ ID NO:8), or any combination thereof, wherein the spacer nucleotide sequence generated by inclusion of TAG, TECH (SEQ ID NO:3), DETECT (SEQ ID NO:4), CRTECH (SEQ ID NO:5), CRTAG (SEQ ID NO:6), CRDETECT (SEQ ID NO:7), CRISPR (SEQ ID NO:8), etc., generates a nucleotide sequence that is non-natural. In some embodiments, the text that is spelled when the spacer tag is translated according to amino acid single-letter code convention may comprise a word, abbreviation, and/or acronym comprising (i) TAG, TECH (SEQ ID NO:3), DETECT (SEQ ID NO:4), CRTECH (SEQ ID NO:5), CRTAG (SEQ ID NO:6), CRDETECT (SEQ ID NO:7) and/or CRISPR (SEQ ID NO:8), and, (ii) an identifiable and/or known moniker such as the name of a company or institution, e.g., NCSTATETECH (SEQ ID NO:11), NCSTATETAG (SEQ ID NO:12). In some embodiments, the text that is spelled when the spacer tag is translated according to amino acid single-letter code convention (e.g., the word, abbreviation, or acronym) may comprise, consist essentially of, or consist of CRISPR (SEQ ID NO:8), CRISPRTECH (SEQ ID NO:9), CRISPRTAG (SEQ ID NO:1), NCSTATE (SEQ ID NO:10), NCSTATETECH (SEQ ID NO:11), NCSTATETAG (SEQ ID NO:12), CRTECH (SEQ ID NO:5), CRTAG (SEQ ID NO:6), DETECT (SEQ ID NO:4), CRDETECT (SEQ ID NO:7), CRISPRDETECT (SEQ ID NO:13), or any combination thereof.

In some embodiments, a synthetic CRISPR spacer polynucleotide of the present invention may be linked at its 5′ end to a repeat sequence to form a repeat-spacer sequence.

A repeat sequence of the present invention (e.g., of a repeat-spacer sequence) may be any natural or non-natural repeat sequence. In some embodiments, a repeat sequence of the present invention may be a natural repeat sequence, e.g., a repeat found within a wild type CRISPR array of a bacterium. Non-limiting examples natural repeat sequences useful with this invention include repeat sequences from a CRISPR array of a Type I, Type II, Type III, Type IV, Type V, Type VI CRISPR system, or any other CRISPR system now known or later identified. Repeat sequences from naturally occurring CRISPRs can be readily identified by using tools such as that of the database CRISPRdb (Grissa et al. (BMC Informatics 8:172 (2007)(doi:10.1186/1471-2105-8-172). This data base is routinely used by researchers to identify CRISPR arrays and their components including the naturally occurring repeated and unique sequences (i.e., the repeat sequences and the spacer sequences). Additionally, classification systems such as that of Marakova et al. (Nat. Rev. Microbiol. 9(6):467-477(2011)) providing system type and subtype for CRISPR-Cas systems may be used as a cross-reference with the CRISPRdb tool of Grissa et al. to assist in the identifying of a natural repeat sequence for any given CRISPR-Cas system.

In some embodiments, a repeat sequence of the present invention may be a non-natural repeat sequence (a synthetic repeat sequence). In some embodiments, a non-natural repeat sequence may be a palindromic sequence having a length of about 20 to about 50 or more nucleotides, e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50, or more nucleotides, wherein the sequence does not share significant sequence identity with any natural repeat sequence found in a wild type CRISPR system. In some embodiments, a non-natural repeat sequence of the present invention may be a non-functional repeat sequence. There may be multiple mechanisms by which a non-natural repeat sequence of the present invention may be non-functional, i.e., non-performing of the biological mechanisms of a native/endogenous repeat sequence, such as described in, e.g., Briner et al., 2014 Mol. Cell 56(2):333-33. For example, in some embodiments, a non-natural repeat sequence of the present invention may be non-functional because it does not interact with the polynucleotides and/or RNAs of the natural CRISPR system in the targeted organism (organism to be tagged).

In some embodiments, a repeat sequence useful with the invention may include a synthetic repeat sequence having a different nucleotide sequence than naturally occurring repeat sequences known in the art. In some embodiments, a repeat sequence may be identical to (i.e., having 100% identity) or substantially identical (e.g., having 80% to 99% identity (e.g., about 80, 81, 82, 83, 84, 85, 86, 87, 88, 89, 90, 91, 92, 93, 94, 95, 96, 97, 98, 99% identity)) to a wild-type (native) repeat sequence. In some embodiments, a synthetic repeat sequence of the present invention may be a non-functional repeat sequence.

Another aspect of the present invention provides a CRISPR array comprising a leader end (e.g., 5′ portion), a trailer end (e.g., 3′ portion), and at least one repeat-spacer sequence of the present invention, wherein the at least one repeat-spacer sequence comprises a 5′ end and a 3′ end and the 3′ end of the repeat spacer sequence is linked to a repeat sequence (e.g., a second repeat sequence to form a repeat-spacer-repeat sequence), which repeat sequence (e.g., second repeat sequence) may comprise the same or a different nucleotide sequence from the repeat sequence of the repeat spacer sequence (e.g., the first repeat).

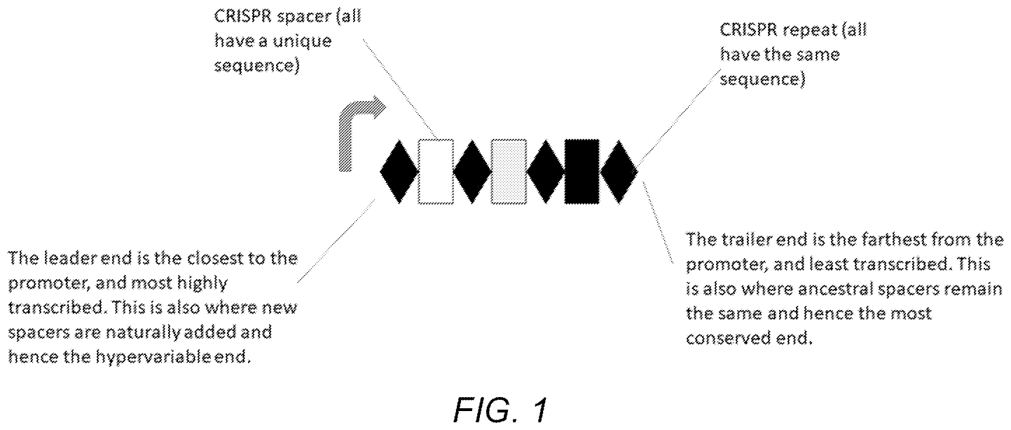

As used herein, the term “trailer end” refers to the 3′ portion of a native CRISPR array. In some embodiments, a trailer end of a CRISPR array may be minimally transcribed (e.g., no transcription of the trailer end or about 1, 2, 3, 4, 5, 6, 7, 8, 9, 10, 11, 12, 13, 14 or 15% transcription as compared to a 5′ portion (leader end) of a CRISPR array). The endogenous function of a CRISPR system of a bacterium comprises an adaptive immune system that “memorizes” previous infections by integrating short sequences of invading genomes (spacers). These short sequences are integrated into a CRISPR array at the 5′ (leader) end. As used herein, the term “leader end” refers to the 5′ portion of a native CRISPR array that is actively transcribed, e.g., nearest to a promoter. As shown in FIG. 1, in a native CRISPR array, the trailer end of is farthest from the promoter and thus, the trailer end is the least transcribed, most conserved portion of the CRISPR array.

In some embodiments, a synthetic CRISPR array of the present invention may comprise two or more repeat-spacer sequences, e.g., may comprise a repeat-spacer sequence of the present invention and further comprise additional natural (e.g., native, endogenous) and/or non-natural repeat-spacer sequences, wherein the two or more repeat-spacer sequences are linked to one another via a repeat sequence (e.g., a repeat-spacer-repeat-spacer-repeat sequence, a repeat-spacer-repeat-spacer-repeat-spacer-repeat sequence, a repeat-spacer-repeat-spacer-repeat-spacer-repeat-spacer-repeat sequence, etc.). In some embodiments, when two or more repeat sequences are present in a CRISPR array, they may comprise the same repeat sequence, may comprise different repeat sequences, or any combination thereof. In some embodiments, each of the two or more repeat sequences in a CRISPR array may comprise, consist essentially of, or consist of the same repeat sequence.

In some embodiments, at least one (e.g., one or more, two or more, three or more, etc.) synthetic repeat-spacer sequence of the present invention may be comprised in (introduced into) an endogenous CRISPR array that is endogenous to a bacterium or archaeon. The endogenous CRISPR array may be from an endogenous CRISPR system of any bacterium or archaeon, such as, but not limited to, Type I, Type II, Type III, Type IV, Type V or Type VI CRISPR system, or any other CRISPR system now known or later identified. In some embodiments, the synthetic CRISPR spacer polynucleotide may be introduced into (located at) the trailer end (e.g., within the 3′ portion) of the endogenous CRISPR array.

A CRISPR array (synthetic and/or native/endogenous) of the present invention may be functional or non-functional. There may be multiple mechanisms by which a synthetic CRISPR array of the present invention may be non-functional, i.e., non-performing of the biological mechanisms of a native/endogenous CRISPR array. For example, in some embodiments, a CRISPR array of the present invention may be non-functional because it may not comprise repeat sequences functional with an endogenous CRISPR system in a bacterium or archaeon of interest. As another example, in some embodiments, a CRISPR array of the present invention may be non-functional because it does not comprise a spacer that targets a sequence comprised in the genome of a bacterium or archaeon of interest (e.g., the spacer is non-targeting). In another example, in some embodiments a CRISPR array of the present invention may be non-functional because it does not comprise a promoter. In another example, in some embodiments a CRISPR array of the present invention may be non-functional because the corresponding endogenous CRISPR system is absent in the target organism, and/or because the corresponding endogenous CRISPR system present in the target organism is itself non-functional.

In some embodiments, an endogenous CRISPR array comprising a synthetic CRISPR spacer polynucleotide of the present invention may be non-functional, e.g., wherein the endogenous CRISPR array is non-transcribing, e.g., does not comprise a promoter; does not comprise repeat sequences functional with an endogenous CRISPR system in a bacterium or archaeon of interest; and/or does not comprise a spacer that targets a sequence comprised in the genome of a bacterium or archaeon of interest. In some embodiments an endogenous CRISPR array of the present invention may be non-functional because components of the endogenous CRISPR system may be absent or non-functional in the target organism. In some embodiments, an endogenous CRISPR array comprising a synthetic CRISPR spacer polynucleotide of the present invention may be functional, wherein the synthetic CRISPR spacer polynucleotide is non-functional, but the endogenous spacer-repeat sequences may be functional, e.g., transcribing, interfering, and/or targeting of a sequence such as that of an invasive genetic element, e.g., a pathogenic phage).

In some embodiments, a synthetic CRISPR spacer polynucleotide and/or synthetic CRISPR array of the present invention may be an “expression cassette” or may be comprised within an expression cassette. As used herein, “expression cassette” means a recombinant nucleic acid construct comprising a polynucleotide of interest (e.g., the synthetic CRISPR spacer polynucleotide and/or synthetic CRISPR array of the invention), wherein said polynucleotide of interest and/or a CRISPR is operably associated with at least one control sequence (e.g., a promoter). Thus, some aspects of the invention provide expression cassettes designed to express the polynucleotides of the invention (e.g., the synthetic CRISPR spacer polynucleotide and/or synthetic CRISPR array of the invention). In some embodiments, the synthetic CRISPR spacer polynucleotide and/or synthetic CRISPR array of the invention explicitly exclude a control sequence (e.g., explicitly exclude a promoter).

An expression cassette comprising a nucleotide sequence of interest may be chimeric, meaning that at least one of its components is heterologous with respect to at least one of its other components. An expression cassette may also be one that is naturally occurring but has been obtained in a recombinant form useful for heterologous expression.

An expression cassette may also optionally include a transcriptional and/or translational termination region (i.e., termination region) that is functional in the selected host cell. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked polynucleotide of interest, may be native to the host cell, or may be derived from another source (i.e., foreign or heterologous to the promoter, to the polynucleotide of interest, to the host, or any combination thereof).

An expression cassette (e.g., recombinant nucleic acid construct(s) of the invention) may also include a nucleotide sequence for a selectable marker, which can be used to select a transformed host cell (e.g., force a cell to acquire and keep an introduced nucleic acid (e.g., expression cassette, vector (e.g., plasmid) comprising the recombinant nucleic acid constructs of the invention)). As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Of course, many examples of suitable selectable markers are known in the art and can be used in the expression cassettes described herein. In some embodiments, a selectable marker useful with this invention includes polynucleotide encoding a polypeptide conferring resistance to an antibiotic. Non-limiting examples of antibiotics useful with this invention include tetracycline, chloramphenicol, and/or erythromycin. Thus, in some embodiments, a polynucleotide encoding a gene for resistance to an antibiotic may be introduced into the organism, thereby conferring resistance to the antibiotic to that organism.

In addition to expression cassettes, the nucleic acid construct and nucleotide sequences described herein may be used in connection with vectors. The term “vector” refers to a composition for transferring, delivering, or introducing a nucleic acid (or nucleic acids) into a cell. A vector comprises a nucleic acid construct comprising the nucleotide sequence(s) to be transferred, delivered, or introduced. Vectors for use in transformation of host organisms are well known in the art. Non-limiting examples of general classes of vectors include but are not limited to a viral vector, a plasmid vector, a phage vector, a phagemid vector, a cosmid vector, a fosmid vector, a bacteriophage, an artificial chromosome, transposon, retrovirus or an Agrobacterium binary vector in double or single stranded linear or circular form which may or may not be self-transmissible or mobilizable. Plasmids useful with the invention may be dependent on the target organism, that is, dependent on where the plasmid is to replicate. Non-limiting examples of plasmids that express in Lactobacillus include pNZ and derivatives, pGK12 and derivatives, pTRK687 and derivatives, pTRK563 and derivatives, pTRKH2 and derivatives, pIL252, and/or pIL253. Additional, non-limiting plasmids of interest include pORI-based plasmids or other derivatives and homologs.

A vector as defined herein can transform a prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). A nucleic acid construct in the vector may be under the control of, and operably linked to, an appropriate promoter or other regulatory elements for transcription in a host cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell. Accordingly, the recombinant nucleic acid constructs of this invention and/or expression cassettes comprising the recombinant nucleic acid constructs of this invention may be comprised in vectors as described herein and as known in the art. Thus, in some embodiments, a vector may comprise a synthetic CRISPR spacer polynucleotide and/or a synthetic CRISPR array of the present invention, e.g., for delivery into a bacterium or archaeon of interest.

Another embodiment of the present invention provides a bacterium or archaeon comprising a synthetic CRISPR spacer polynucleotide and/or a synthetic CRISPR array of the present invention, and/or a vector comprising the same (a synthetic CRISPR spacer polynucleotide and/or synthetic CRISPR array of the present invention).

### Methods of Labeling and Detecting Bacteria of Interest

The compositions (e.g., synthetic CRISPR spacer polynucleotides and/or synthetic CRISPR arrays) of the present invention may be used, for example, in methods for labeling and/or identifying bacterial cells.

Accordingly, the synthetic polynucleotides (e.g., synthetic CRISPR spacer polynucleotide and/or array) of the invention may be introduced into a cell of an organism. In some embodiments, the recombinant nucleic acid constructs of the invention may be stably or transiently introduced into a cell of a bacterium of interest for the purpose of labeling (e.g., tagging) the cell of a bacterium or archaeon of interest, optionally for labeling (e.g., tagging) an endogenous CRISPR array in the cell of a bacterium or archaeon of interest.

Thus, one aspect of the present invention provides a method of labeling a bacterial cell, comprising: (a) introducing into an endogenous/native CRISPR array of the bacterial cell a synthetic CRISPR spacer polynucleotide of the present invention (e.g., a repeat-spacer sequence of the present invention; e.g., a non-functional repeat-spacer sequence of the present invention) or a vector comprising the synthetic CRISPR spacer polynucleotide, the endogenous CRISPR array comprising a leader end (5′ portion) and a trailer end (3′ portion), wherein the synthetic CRISPR spacer polynucleotide is introduced into the CRISPR array; and/or (b) introducing into the bacterial cell a synthetic CRISPR array of the present invention or a vector comprising the synthetic CRISPR array; thereby labeling the bacterial cell.

Another aspect of the present invention provides a method of detecting a bacterial cell, comprising: (a) introducing into an endogenous/native CRISPR array of the bacterial cell (i) a synthetic CRISPR spacer polynucleotide of the present invention (e.g., a repeat-spacer sequence of the present invention) or a vector comprising the synthetic CRISPR spacer polynucleotide of the present invention, the endogenous CRISPR array comprising a leader end (5′ portion) and a trailer end (3′ portion); and/or (ii) introducing into the bacterial cell a synthetic CRISPR array of the present invention or a vector comprising the synthetic CRISPR array, thereby labeling the bacterial cell; and (b) detecting the presence of the synthetic polynucleotide, thereby detecting the bacterial cell.

A synthetic CRISPR spacer polynucleotide may be introduced into any portion (3′ portion, 5′ portion) of a native CRISPR array in the bacterial cell. In some embodiments, a synthetic CRISPR spacer polynucleotide may be inserted into the middle of a native CRISPR array, optionally wherein the synthetic CRISPR spacer polynucleotide is non-functional. In some embodiments, a synthetic CRISPR spacer polynucleotide may be inserted into the 5′ portion (leader end) of a native CRISPR array, optionally wherein the synthetic CRISPR spacer polynucleotide is non-functional. In some embodiments, a synthetic CRISPR spacer polynucleotide may be inserted into the 3′ portion (trailer end) of a native CRISPR array, optionally wherein the synthetic CRISPR spacer polynucleotide is non-functional. In some embodiments, a synthetic CRISPR spacer polynucleotide may be inserted at the 3′ end of a native CRISPR array (i.e., inserted as a spacer-repeat sequence at the 3′ end immediately adjacent the most distal repeat sequence), optionally wherein the synthetic CRISPR spacer polynucleotide is non-functional.

Thus, in some embodiments, the present invention provides a method of labeling a bacterial cell, comprising: (a) introducing into an endogenous/native CRISPR array of the bacterial cell a synthetic CRISPR spacer polynucleotide of the present invention (e.g., a repeat-spacer sequence of the present invention) or a vector comprising the synthetic CRISPR spacer polynucleotide, the endogenous CRISPR array comprising a leader end (5′ portion) and a trailer end (3′ portion), wherein the synthetic CRISPR spacer polynucleotide is introduced into/at the trailer end of the CRISPR array; and/or (b) introducing into the bacterial cell a synthetic CRISPR array of the present invention or a vector comprising the synthetic CRISPR array; thereby labeling the bacterial cell.

Similarly, in some embodiments, the present invention provides a method of detecting a bacterial cell, comprising: (a) introducing into an endogenous/native CRISPR array of the bacterial cell (i) a synthetic CRISPR spacer polynucleotide of the present invention (e.g., a repeat-spacer sequence of the present invention) or a vector comprising the synthetic CRISPR spacer polynucleotide of the present invention, the endogenous CRISPR array comprising a leader end (5′ portion) and a trailer end (3′ portion), wherein the synthetic CRISPR spacer polynucleotide is introduced into/at the trailer end of the CRISPR array; and/or (ii) introducing into the bacterial cell a synthetic CRISPR array of the present invention or a vector comprising the synthetic CRISPR array, thereby labeling the bacterial cell; and (b) detecting the presence of the synthetic polynucleotide, thereby detecting the bacterial cell.

In some embodiments of the present methods, the endogenous CRISPR array is functional and the synthetic CRISPR spacer polynucleotide that is introduced is not functional (e.g., non-transcribing, non-interfering and/or non-targeting, etc.).

In some embodiments, the methods of the present invention further comprise a step of determining that there is no target sequence in the targeted bacterium (bacterium of interest, e.g., bacterium to be targeted) that is complementary to the CRISPR tag. In some embodiments, the methods of the present invention further comprise a step of determining that there is no target sequence flanked by (e.g., adjacent to) a PAM in the genome of the targeted bacterium (bacterium of interest, e.g., bacterium to be targeted) that is complementary to the CRISPR tag.

“Introducing,” “introduce,” “introduced” (and grammatical variations thereof) in the context of a nucleotide sequence (e.g., a synthetic CRISPR spacer polynucleotide and/or synthetic CRISPR array of the present invention) and a cell of an organism means presenting the nucleotide sequence to the host organism or cell of said organism (e.g., host cell) in such a manner that the nucleotide sequence gains access to the interior of a cell and includes such terms as “transformation,” “transfection,” and/or “transduction.” Transformation may be electrical (electroporation and electrotransformation), or chemical (with a chemical compound, and/or though modification of the pH and/or temperature in the growth environment. Where more than one nucleotide sequence is to be introduced these nucleotide sequences can be assembled as part of a single polynucleotide or nucleic acid construct, or as separate polynucleotide or nucleic acid constructs, and can be located on the same or different expression constructs or transformation vectors. Accordingly, these polynucleotides can be introduced into cells in a single transformation event, in separate transformation events, or, for example, they can be incorporated into an organism by conventional breeding or growth protocols.

Thus, in some aspects of the present invention one or more synthetic CRISPR spacer polynucleotide and/or synthetic CRISPR array of this invention may be introduced into a host bacterium.

The terms “transformation,” “transfection,” and “transduction” as used herein refer to the introduction of a heterologous nucleic acid into a cell. Such introduction into a cell may be stable or transient. Thus, in some embodiments, a host cell or host organism is stably transformed with a nucleic acid construct of the invention. In other embodiments, a host cell or host organism is transiently transformed with a recombinant nucleic acid construct of the invention.

As used herein, the term “stably introduced” means that an introduced polynucleotide is stably incorporated into the genome of the cell, and thus the cell is stably transformed with the polynucleotide. When a nucleic acid construct is stably transformed and therefore integrated into a cell, the integrated nucleic acid construct is capable of being inherited by the progeny thereof, more particularly, by the progeny of multiple successive generations. In some embodiments, the term “stably introduced” means that an introduced protein-RNA complex of the invention is stably maintained in the cell into which it is introduced.

“Transient transformation” in the context of a polynucleotide means that a polynucleotide is introduced into the cell and does not integrate into the genome of the cell or is not otherwise maintained by the cell.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant, a mammal, an insect, an archaea, a bacterium, and the like). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

Accordingly, in some embodiments, the synthetic CRISPR spacer polynucleotide(s) and/or synthetic CRISPR array(s) of the present invention are stably incorporated into the genome of the host organism. In some embodiments, a synthetic CRISPR spacer polynucleotide and/or synthetic CRISPR array of the present invention is maintained in a bacterial cell as an extrachromosomal element (e.g., a plasmid). In some embodiments, a synthetic CRISPR spacer polynucleotide and/or synthetic CRISPR array of the present invention is incorporated into the chromosome of the bacterial cell. In some embodiments, when a non-natural CRISPR of the invention is incorporated into an endogenous array of the host bacterium, the non-natural CRISPR is incorporated into the endogenous array in the 3′ portion of endogenous CRISPR array (trailer end), near the 3′ end of the endogenous CRISPR array and/or adjacent to and 3′ of the 3′ end of the last repeat of the endogenous CRISPR array, e.g., distal to the leader end, e.g., downstream of the promoter to such a distance that the non-natural CRISPR is minimally or not transcribed.

A synthetic CRISPR spacer polynucleotide and/or synthetic CRISPR array of the invention may be introduced into a cell by any method known to those of skill in the art. Exemplary methods of transformation or transfection include biological methods using viruses and bacteria (e.g., Agrobacterium), physicochemical methods such as electroporation, floral dip methods, particle or ballistic bombardment, microinjection, whiskers technology, pollen tube transformation, calcium-phosphate-mediated transformation, nanoparticle-mediated transformation, polymer-mediated transformation including cyclodextrin-mediated and polyethyleneglycol-mediated transformation, sonication, infiltration, as well as any other electrical, chemical, physical (mechanical) and/or biological mechanism that results in the introduction of nucleic acid into a cell, including any combination thereof.

In some embodiments of the invention, transformation of a cell comprises nuclear transformation. In other embodiments, transformation of a cell comprises plastid transformation (e.g., chloroplast transformation). In still further embodiments, the recombinant nucleic acid construct of the invention can be introduced into a cell via conventional breeding techniques.

Procedures for transforming prokaryotic organisms are well known and routine in the art and are described throughout the literature (See, for example, Jiang et al. 2013. Nat. Biotechnol. 31:233-239; Ran et al. Nature Protocols 8:2281-2308 (2013))

A nucleotide sequence therefore can be introduced into a host organism or its cell in any number of ways that are well known in the art. The methods of the invention do not depend on a particular method for introducing one or more nucleotide sequences into the organism, only that they gain access to the interior of at least one cell of the organism. In some embodiments, a nucleotide sequence of the present invention (e.g., synthetic CRISPR spacer polynucleotide and/or synthetic CRISPR array of the present invention) may be incorporated into the genome of a bacterium of interest. Mechanisms for introducing a nucleotide sequence into a genome are known in the art, including, but not limited to, insertion via homology directed repair (HDR) (homologous recombination). Thus, in some embodiments, the synthetic CRISPR spacer polynucleotide is inserted into/at the trailer end (3′ portion) of the endogenous/native CRISPR array, for example, via HDR.

In some embodiments, a method of labeling a bacterial cell of the present invention may further comprise detecting the presence of the synthetic polynucleotide. Detecting the presence of the synthetic polynucleotide may be performed via any known mechanism in the art that would be useful toward determining the text encoded by the synthetic polynucleotide, such as, but not limited to, via polymerase chain reaction (PCR) and/or sequencing (e.g., Sanger sequencing, whole-genome sequencing, 16S sequencing, internal transcribed spacer (ITS) RNA sequencing, metagenomic (shotgun) sequencing, and/or any other nucleotide sequencing technique). In some embodiments, the sequence of the synthetic CRISPR spacer polynucleotide, the sequence of the spacer tag, and/or the sequence of a repeat sequence linked to the 5′ end and/or 3′ end of the synthetic CRISPR spacer polynucleotide may be a primer, e.g., for the use of PCR and/or sequencing.

The compositions and methods of the present invention may be applied to any bacteria of interest. A bacterial cell useful with this invention can be, but is not limited to, a lactic acid bacterial cell, a probiotic bacterial cell, an industrial fermentation bacterial cell, a microbiome bacterial cell, a biotherapeutic bacterial cell, pathogenic bacterial cell, a commensal bacterial cell, a spoilage bacterial cell (e.g., food spoilage), and/or any combination thereof.

In some embodiments, the synthetic CRISPR spacer polynucleotide is maintained in a bacterial cell as an extrachromosomal element (e.g., a plasmid).

In some embodiments, a bacterial cell can be in a sample. The sample may be, but is not limited to, a sample from a bacterial culture, a subject (e.g., an animal subject, a human subject), a plant, a food, soil, water, and/or any combination thereof. In some embodiments, the sample from the subject is in vivo and/or ex vivo (e.g., an in vivo and/or ex vivo microbiome sample).

A “subject” of the invention includes any animal comprising a microbiome. Such a subject is generally a mammalian subject (e.g., a laboratory animal such as a rat, mouse, guinea pig, rabbit, primates, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, etc.) or a human.

A bacterial cell may be any bacterial cell or bacterial population for which tagging/labeling, tracking, and/or identifying the bacterium and/or archaeon is desirable. Such bacteria or archaea include, for example, commensal, probiotic or other commercially useful bacteria or strain of bacteria. In some embodiments, the bacterial cell is a cell from a Clostridium spp., a Erysipelatoclostridium spp., a Lactococcus spp., a Streptococcus spp., a Klebsiella spp., a Propionibacterium spp., a Cutibacterium spp., a Lactobacillus spp., a Pseudomonas spp., a Faecalibacterium spp., an Akkermansia spp., a Bifidobacterium spp., a Roseburia spp., a Bacteroides spp., a Collinsella spp., a Dorea spp., a Bacillus spp., a Eubacterium spp., a Blautia spp., an E. coli spp., or a Clostridioides spp. In some embodiments, the bacterial cell is from a commensal bacterial species, optionally a Lactobacillus spp., or a Bifidobacterium spp. or a Clostridium spp. In some embodiments, bacteria useful with the invention include, but are not limited to, those in the species of L. acidophilus, L. casei, L. paracasei, L. crispatus, L. gasseri, L. plantarum, L. rhamnosus, L. salivarius, L. fermentum, L. reuteri, L. johnsonii, B. longum, B. lactis, B. infantis, or any combination thereof.

A bacterial cell for use with this invention may be a single cell or a cell within a population of bacterial cells of the same species or strain or may be a cell within a population comprising a mixture of two or more bacterial species or strains. In some embodiments, the methods of this invention may be carried out on a portion of a population of bacterial cells. As used herein, “a portion of the population of cells” means at least one cell of a population of two or more cells (e.g., 2, 3, 4, 5, 6, 7, 8, 9, 10, or more cells, e.g., 10², 10³, 10⁴, 10⁵, 10⁶, 10⁷, 10⁸, 10⁹, 10¹⁰ or more cells).

In some embodiments, when a bacterial cell for which tagging/labeling, tracking, and/or identifying does not comprise an endogenous CRISPR-Cas system that is functional and/or compatible with the recombinant CRISPR nucleic acids (e.g., a synthetic CRISPR array, a synthetic CRISPR spacer polynucleotide) of the invention.

In some embodiments, the invention further comprises recombinant bacterial cells produced by the methods of the invention, comprising the synthetic polynucleotides of the invention (e.g., synthetic CRISPR array, a synthetic CRISPR spacer polynucleotide), and/or a vector, plasmid, and/or bacteriophage comprising the synthetic polynucleotides of the invention, and/or the genome modifications generated by the methods of the invention.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1: CRISPR Spacers as Tags for Bacteria of Interest

In a native (endogenous) CRISPR array, the leader end (5′ portion) is the closest to the promoter, and most highly transcribed. This is also where new spacers are naturally added and is hence the hypervariable end of the CRISPR array. The trailer end (3′ portion) is the farthest from the promoter, and least transcribed. This is also where ancestral spacers remain the same and hence this is the most conserved end, as overviewed in FIG. 1. The present invention provides synthetic CRISPR spacer polynucleotides (synthetic spacers) that encode combinations of amino acids, to serve as a synthetic genetic tag for the end user to detect.

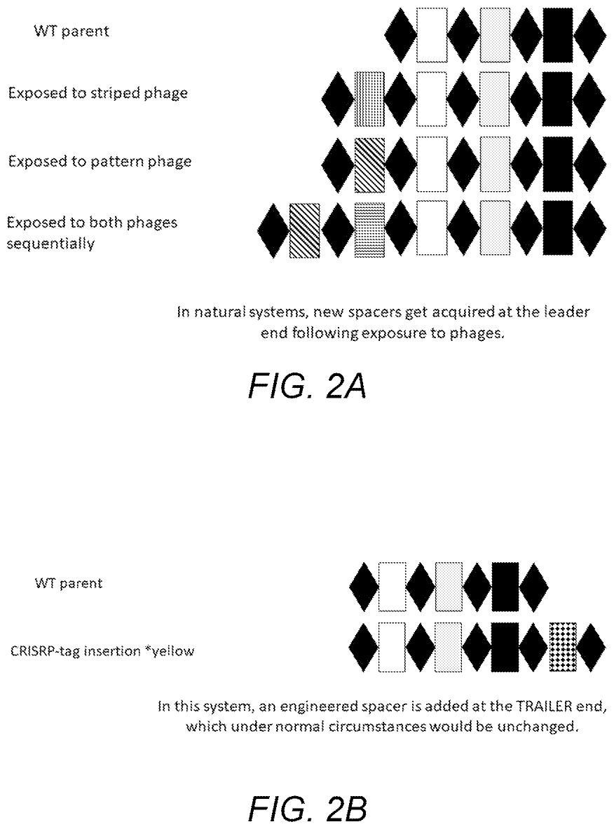

FIG. 2A provides a schematic of a natural CRISPR system, wherein the wild type (WT) parent bacterium may be exposed to a succession of infectious bacteriophages. Upon exposure to phage, new spacers (as “repeat-spacer” units) are inserted into an endogenous CRISPR array at the leader end (5′ transcribed portion). Conversely, as shown in FIG. 2B, the present invention provides synthetic CRISPR spacer polynucleotides comprising CRISPR tags (“CRISPR-tag”), which are introduced into/at the trailer end of a CRISPR array (for example, an endogenous/native CRISPR array of a bacterium of interest). In this system, an engineered spacer is added at the trailer end of the endogenous array, which under normal/wild-type circumstances would remain unchanged in sequence and minimally to non-transcribed.

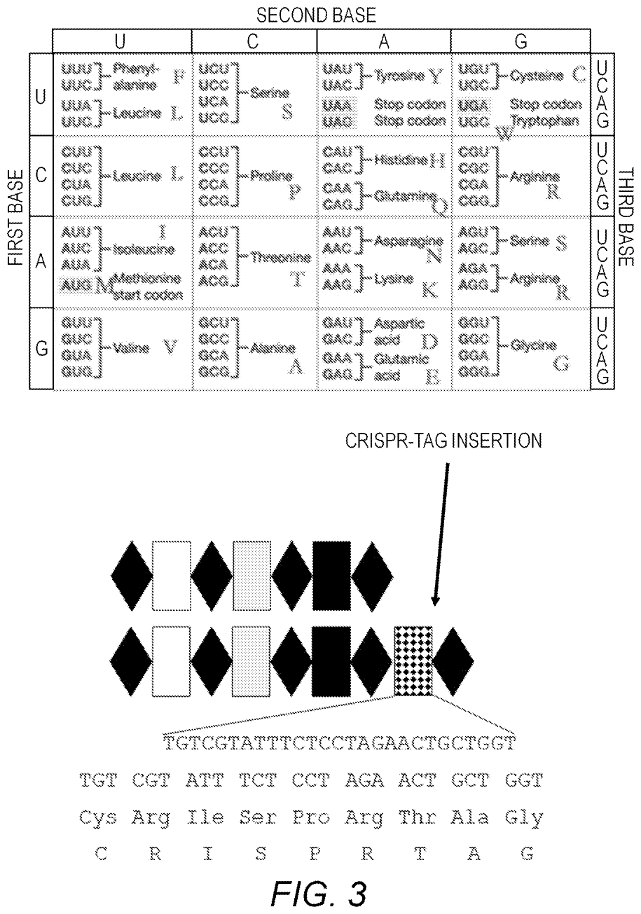

The CRISPR tags of the present invention are designed to contain a polynucleotide that encode an amino acid sequence which, when translated according to amino acid single-letter code convention, spell a text (for example, “CRISPRTAG”; SEQ ID NO:1, FIG. 3). These tags may be used to genetically tag an organism of interest, which may then be targeted for detection, for example, by sequencing and/or by polymerase chain reaction (PCR)-based amplification, using designed primers wherein one primer binds to the tag and a second primer binds to the flanking repeat sequence.

Tagging of a bacterium of interest using the methods and compositions of the present invention may be achieved in multiple ways, including but not limited to, by inserting a spacer within an endogenous CRISPR-Cas system, and/or by introducing an exogenous synthetic CRISPR array into a bacterium (e.g., into the genome of the bacterium), which bacterium may or may not otherwise be devoid of such a CRISPR array. In addition, in any of these scenarios, introduction of an engineered CRISPR spacer comprising a CRISPR tag may be achieved with either a natural repeat sequence or an engineered synthetic repeat sequence.

### Example 2

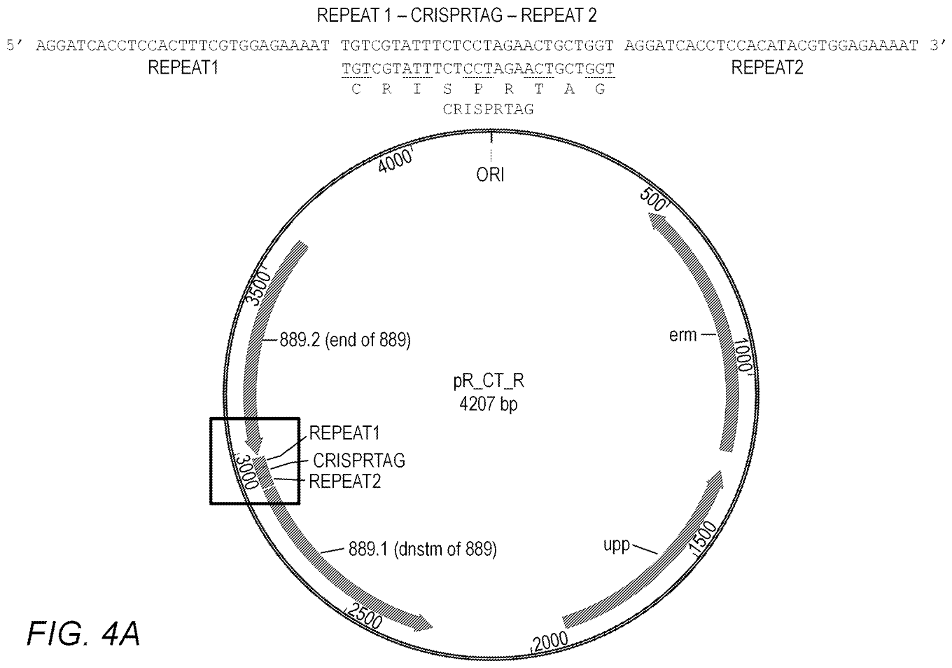

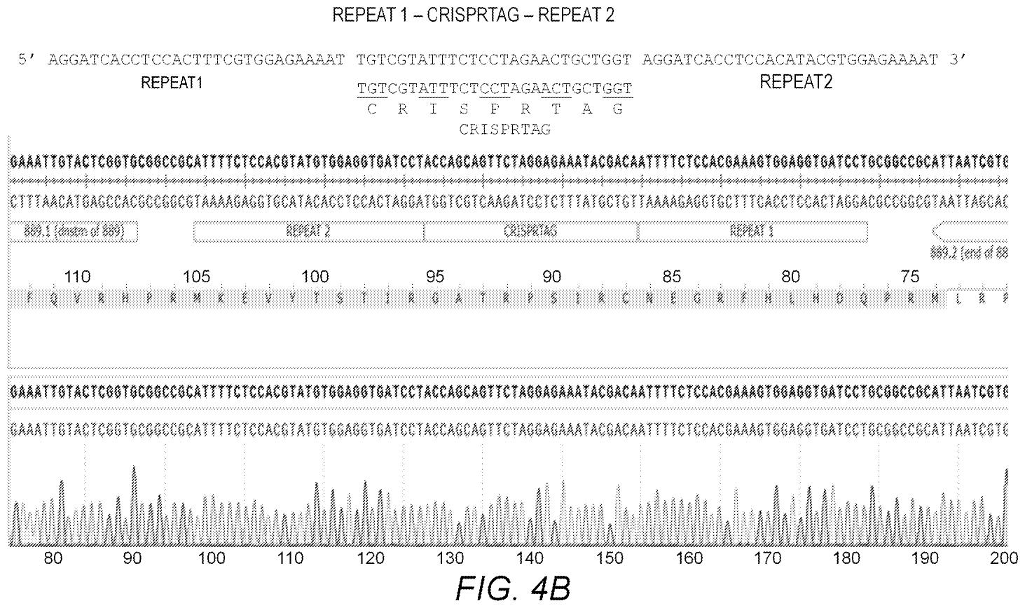

FIGS. 4A-4B display a DNA sequence designed to generate a synthetic array comprised of two repeats (Repeat1, Repeat2) flanking a spacer DNA sequence encoding a peptide, wherein the spacer encodes an amino acid sequence that spells out “CRISPRTAG” (SEQ ID NO:1) using the codon table and showcasing amino acids in their single letter code (e.g., C=Cys=Cysteine; R=Arg=Arginine; I=Ile=Isoleucine; S=Ser=Serine; P=Pro=Proline; R=Arg=Arginine; T=The =Threonine; A=Ala=Alanine; G=Gly=Glycine). FIG. 4A also shows a schematic (map) of a corresponding plasmid, pR_CR_R, generated to encode the above-described sequence, wherein the plasmid has an R (repeat) followed by a CT (CRISPRTAG; SEQ ID NO:1), followed by another R (repeat) (boxed inset). Also shown in the plasmid map are other example components of the example plasmid. FIG. 4B shows the sequence region of the boxed inset of FIG. 4A, indicating the sequencing electropherogram produced, showing the generated sequence in a cellular context with the sequence above.

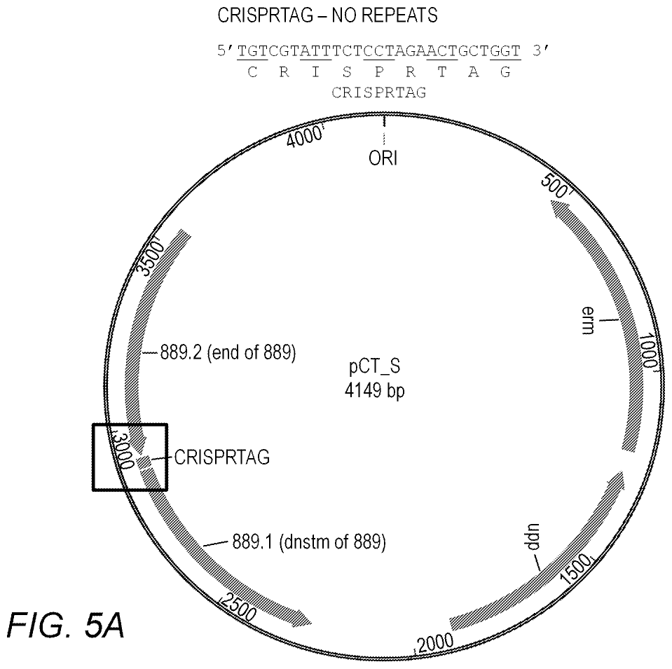

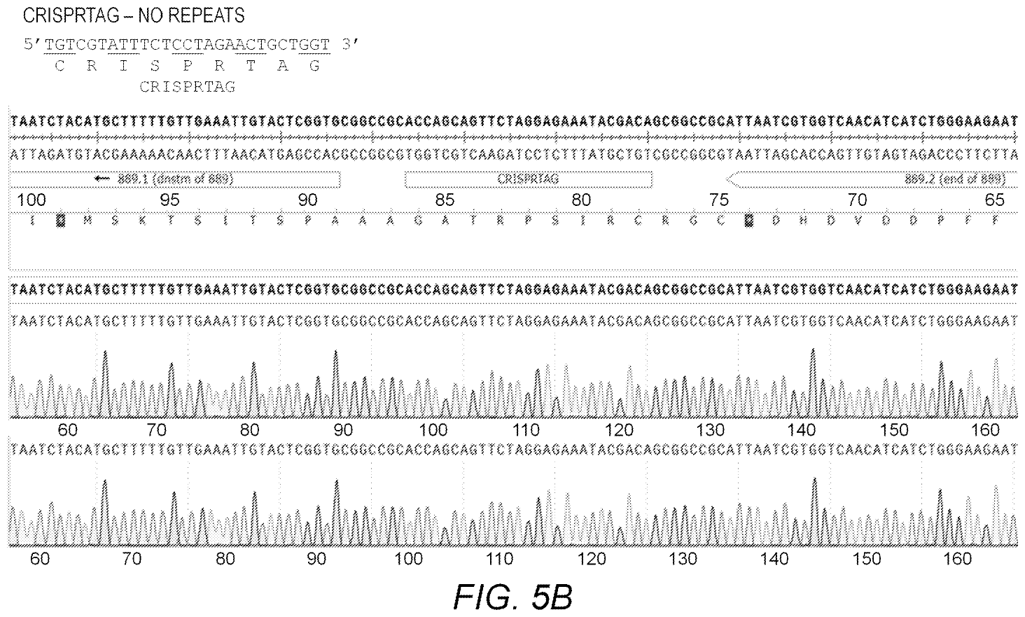

Similarly, FIGS. 5A-5B show display a DNA sequence designed to generate a synthetic CRISPRtag comprised of a spacer DNA sequence encoding a peptide, wherein the spacer encodes an amino acid sequence that spells out “CRISPRTAG” (SEQ ID NO:1) using the codon table and showcasing amino acids in their single letter code (e.g., C=Cys=Cysteine; R=Arg=Arginine; I=Ile=Isoleucine; S=Ser=Serine; P=Pro=Proline; R=Arg=Arginine; T=The =Threonine; A=Ala=Alanine; G=Gly=Glycine). FIG. 5A also shows a schematic (map) of a corresponding plasmid, pCT_S, generated to encode the above-described sequence, a single CT (CRISPRTAG; SEQ ID NO:1). Also shown in the plasmid map are other example components of the example plasmid. FIG. 5B shows the sequence region of the boxed inset of FIG. 5A, indicating the sequencing electropherogram produced, showing the generated sequence in a cellular context with the sequence above.

The foregoing is illustrative of the present invention, and is not to be construed as limiting thereof. The invention is defined by the following claims, with equivalents of the claims to be included therein.

1. A synthetic CRISPR spacer polynucleotide having a 5′ end and a 3′ end and comprising a spacer tag having a length of about 20 to about 50 nucleotides, which when translated according to amino acid single-letter code convention spells a text, wherein the synthetic CRISPR spacer polynucleotide is non-functional.

2. The synthetic CRISPR spacer polynucleotide, wherein the synthetic CRISPR spacer polynucleotide comprises a length of about 20 nucleotides to about 100 nucleotides.

3. The synthetic CRISPR spacer polynucleotide, wherein when the synthetic CRISPR spacer polynucleotide comprises a length greater than the length of the spacer tag, and wherein the spacer tag is located at either the 5′ end, at the 3′ end, or any position within the synthetic CRISPR spacer polynucleotide.

4. The synthetic CRISPR spacer polynucleotide of claim 1, wherein the non-functional synthetic CRISPR spacer polynucleotide is non-transcribable, non-interfering and/or non-targeting.

5. The synthetic CRISPR spacer polynucleotide of claim 1, wherein the text is a word, an abbreviation, and/or an acronym.

6. The synthetic CRISPR spacer polynucleotide of claim 5, wherein the word, abbreviation or acronym comprises TAG, TECH (SEQ ID NO:3), DETECT (SEQ ID NO:4), CRTECH (SEQ ID NO:5), CRTAG (SEQ ID NO:6), CRDETECT (SEQ ID NO:7), CRISPR (SEQ ID NO:8), or any combination thereof.

7. The synthetic CRISPR spacer polynucleotide of claim 5, wherein the word, abbreviation or acronym is CRISPR (SEQ ID NO:8), CRISPRTECH (SEQ ID NO:9), CRISPRTAG (SEQ ID NO:1), NCSTATE (SEQ ID NO:10), NCSTATETECH (SEQ ID NO:11), NCSTATETAG (SEQ ID NO:12), CRTECH (SEQ ID NO:5), CRTAG (SEQ ID NO:6), DETECT (SEQ ID NO:4), CRDETECT (SEQ ID NO:7), CRISPRDETECT (SEQ ID NO:13), or any combination thereof.

8. The synthetic CRISPR spacer polynucleotide of claim 1, linked at its 5′ end to a repeat sequence to form a repeat-spacer sequence.

9. The synthetic CRISPR spacer polynucleotide of claim 8, wherein the repeat sequence of the repeat spacer sequence is a natural repeat sequence.

10. The synthetic CRISPR spacer polynucleotide of claim 8, wherein the repeat sequence of the repeat spacer sequences is a non-natural repeat.

11. A synthetic CRISPR array comprising a leader end, a trailer end, and at least one repeat-spacer sequence of claim 8, wherein the at least one repeat-spacer sequence comprises a 5′ end and a 3′ end and the 3′ end of the repeat spacer sequence is linked to a repeat sequence, wherein the synthetic CRISPR array is non-functional.

12. The synthetic CRISPR array of claim 11, wherein the synthetic CRISPR array further comprises two or more repeat-spacer sequences of claim 8, wherein the two or more two repeat-spacer sequences are linked to one another via a repeat sequence.

13. The synthetic CRISPR array of claim 11, wherein at least one synthetic repeat-spacer of claim 8 is comprised in an endogenous CRISPR array that is endogenous to a bacterium or archaeon, wherein the endogenous CRISPR array is from a Type I, Type II, Type III, Type IV, Type V or Type VI CRISPR system.

14. The synthetic CRISPR array of claim 13, wherein the synthetic CRISPR spacer polynucleotide is located at the trailer end of the endogenous CRISPR array.

15-16. (canceled)

17. A vector comprising the synthetic CRISPR spacer polynucleotide of claim 1, optionally wherein the vector is a plasmid, a phagemid, a transposon, or a bacteriophage.

18. (canceled)

19. A bacterium comprising the synthetic CRISPR spacer polynucleotide of claim 1.

20. A method of labeling a bacterial cell, comprising:
(a) introducing into an endogenous/native CRISPR array of the bacterial cell a synthetic CRISPR spacer polynucleotide of claim 8, the endogenous CRISPR array comprising a leader end and a trailer end, wherein the synthetic CRISPR spacer polynucleotide is introduced into/at the trailer end of the CRISPR array;
thereby labeling the bacterial cell.

21-24. (canceled)

25. A method of detecting a bacterial cell, comprising:
(a) introducing into an endogenous/native CRISPR array of the bacterial cell a synthetic CRISPR spacer polynucleotide of claim 8, the endogenous CRISPR array comprising a leader end and a trailer end, wherein the synthetic CRISPR spacer polynucleotide is introduced into/at the trailer end of the CRISPR array; and
(b) detecting the presence of the synthetic polynucleotide, thereby detecting the bacterial cell.

26-37. (canceled)

38. A method of labeling a bacterial cell, comprising:
introducing into the bacterial cell a synthetic CRISPR array of claim 11;
thereby labeling the bacterial cell.

39. A method of detecting a bacterial cell, comprising:
(a) introducing into the bacterial cell a synthetic CRISPR array of claim 11, thereby labeling the bacterial cell; and
(b) detecting the presence of the synthetic polynucleotide, thereby detecting the bacterial cell.


SEQUENCE LISTING (13 sequences)

SEQ ID NO: 1 - protein, 9 aa, Artificial Sequence
  spacer tag
CRISPRTAG

SEQ ID NO: 2 - DNA, 27 bp, Artificial Sequence
  spacer tag
tgtcgtatttctcctagaactgctggt

SEQ ID NO: 3 - protein, 4 aa, Artificial Sequence
  spacer tag
TECH

SEQ ID NO: 4 - protein, 6 aa, Artificial Sequence
  spacer tag
DETECT

SEQ ID NO: 5 - protein, 6 aa, Artificial Sequence
  spacer tag
CRTECH

SEQ ID NO: 6 - protein, 5 aa, Artificial Sequence
  spacer tag
CRTAG

SEQ ID NO: 7 - protein, 8 aa, Artificial Sequence
  spacer tag
CRDETECT

SEQ ID NO: 8 - protein, 6 aa, Artificial Sequence
  spacer tag
CRISPR

SEQ ID NO: 9 - protein, 10 aa, Artificial Sequence
  spacer tag
CRISPRTECH

SEQ ID NO: 10 - protein, 7 aa, Artificial Sequence
  spacer tag
NCSTATE

SEQ ID NO: 11 - protein, 11 aa, Artificial Sequence
  spacer tag
NCSTATETECH

SEQ ID NO: 12 - protein, 10 aa, Artificial Sequence
  spacer tag
NCSTATETAG

SEQ ID NO: 13 - protein, 12 aa, Artificial Sequence
  spacer tag
CRISPRDETECT